(19) United States
(12) Patent Application Publication
(10) Pub. No.: US 2009/0221953 A1
(43) Pub. Date: Sep. 3, 2009

(54) COMPOSITION COMPRISING PROTEIN-LIPOSOME COMPLEX FOR IONTOPHORESIS

(76) Inventors: Kazuaki Kajimoto, Sapporo (JP); Masahiko Yamamoto, Sapporo (JP); Kentaro Kogure, Kyoto (JP); Hideyoshi Harashima, Sapporo (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 12/392,885

(22) Filed: Feb. 25, 2009

Related U.S. Application Data
(60) Provisional application No. 61/088,939, filed on Aug. 14, 2008.

(30) Foreign Application Priority Data
Feb. 26, 2008 (JP) 2008044839

(51) Int. Cl. A61N 1/30; A61K 9/127; A61K 39/00

(52) U.S. Cl. 604/20; 424/450; 424/184.1

(57) ABSTRACT

Provided is a composition for iontophoresis comprising a negatively-charged protein-liposome complex, in which the protein-liposome complex is formed of a negatively-charged protein and a cationic liposome. Such may provide a composition capable of efficiently delivering a protein having a large molecular weight intradermally and inducing an immune response effectively by iontophoresis.

### CROSS REFERENCE TO RELATED APPLICATIONS

This application claims benefit under 35 U.S.C. 119(e) of U.S. Provisional Patent Application Ser. No. 61/088,939, filed Aug. 14, 2008, entitled “Iontophoresis Composition Containing Protein-Liposome Compound”, which is incorporated herein by reference in its entirety.

### BACKGROUND

1. Technical Field

The present application relates to compositions useful in methods of intradermally administering a protein to an organism by iontophoresis, wherein the compositions comprise a negatively-charged protein-liposome complex.

2. Description of the Related Art

The epidermal layer is rich in an antigen-presenting cell (e.g., Langerhans cell) which play an important role in the immune system. An intradermal vaccine can deliver an antigen to a target cell, such as an antigen-presenting cell, more efficiently than other administration routes.

Intradermal injection has been generally used as a method of intradermally administering an antigen. In recent years, methods of delivering an antigen by forming a small pore on a skin by physical force such as a force of a needle, pressure, or electricity, e.g., a microneedle, a jet injector, or an electroporation, have been developed. However, the administration methods developed up to the present to deliver an antigen intradermally are generally associated with pain to the patient due, for example, to the requirement that the horny layer of the skin must be punctured to some extent (e.g., when using a jet injector) or to the requirement that a high voltage be applied to the skin of the patient (e.g., when using electroporation). In addition, it is difficult to induce a sufficient immune response by administering an antigen by a microneedle, a jet injector or electroporation as compared to administering an antigen by intradermal injection. Thus, the methods of intradermally administering a vaccine developed up to the present have problems in both safety and effectiveness.

On the other hand, a method of introducing, i.e. permeating, an ionic drug through the skin or the mucosa (herein collectively referred to as “skin”) of an organism by applying an electromotive force to the ionic drug, which has been applied in a predetermined portion to the skin of an organism, is called iontophoresis (see, for example, JP 63-35266 A). In recent years, iontophoresis has been disclosed as a noninvasive and safe method for administering a drug to an organism.

In iontophoresis, an ionic compound having a positive charge is generally driven, i.e., transported, through and/or into the skin of an organism by repulsion by the anode side of the electromotive force. On the other hand, an ionic compound having a negative charge is generally driven, i.e. transported, through and/or into the skin of an organism by repulsion by the cathode side of the electromotive force.

For example, Marro, D. et al., Pharmaceutical Research, 2001 December, Vol. 18, No. 12, pp. 1701-1708 reports that in the case where a drug is administered from a cathode side by iontophoresis, administration efficiency of the drug decreases due to ion infiltration flow (flow of water) from the inside to the outside of a skin; and in the case where a drug is administered from an anode side, the drug is efficiently delivered intradermally by an electric force and the ion infiltration flow.

In addition, it has been suggested in PCT/JP2007/071368 that a drug may be efficiently delivered intradermally by administering a cationic liposome encapsulating the drug from an anode side of an iontophoresis device. However, from experiments by the applicants of the present application, it has been clarified that it is difficult to deliver an antigen protein intradermally and efficiently even by administering a cationic liposome encapsulating a large antigen protein from the anode side by iontophoresis.

### BRIEF SUMMARY

Disclosed herein are methods of intradermally administering to an organism a protein having a large molecular weight efficiently by iontophoresis, wherein the protein effectively induces an immune response in the organism.

The applicants have found that when a negatively-charged complex formed of a protein having antigenicity and a cationic liposome is administered to an organism by iontophoresis, the intradermal delivery of the protein may be remarkably promoted and the immune response may be effectively induced. Embodiments of the present application are based on such findings.

Accordingly, one embodiment of the application provides a composition for iontophoresis that is capable of efficiently delivering a protein intradermally and effectively inducing an immune response.

Such a composition for iontophoresis comprises a protein-liposome complex which is charged negatively, wherein the protein-liposome complex is formed of a negatively-charged protein and a cationic liposome.

Such a composition for iontophoresis may allow even a protein having a relatively large molecular weight, such as an antigen protein, to be efficiently delivered intradermally by iontophoresis and the immune response can thus be effectively induced. Further, such a composition for iontophoresis may be capable of remarkably inducing the immune response of an organism when used together with an adjuvant.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a schematic drawing showing an iontophoresis device used for administering an OVA-liposome complex in an in vivo test according to one illustrated embodiment.

FIG. 2A is a photograph of a frozen skin sample of a rat after OVA-liposome complex administration with an inverted confocal laser scanning microscope.

FIG. 2B is a photograph of a frozen skin sample of a rat after OVA-encapsulated liposome administration with an inverted confocal laser scanning microscope.

FIG. 3 shows a result of ELISA relating to the production amount of an anti-OVA antibody in serum of a rat to which an OVA-liposome complex or an OVA-encapsulated liposome was administered.

FIG. 4 shows a result of ELISA relating to the production amount of an anti-OVA antibody in serum of a rat to which monophospholipid A (MPL) and an OVA-liposome complex were administered together.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with intradermal delivery devices and iontophoresis have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

As used herein and in the claims, the term “cationic liposome” means a liposome having a net positive charge in a selected pH such as a physiological pH.

As used herein and in the claims, the term “cationic lipid” means a lipid having a net positive charge in a selected pH such as a physiological pH.

As used herein and in the claims, the term “fatty acid” may be a saturated or unsaturated, and a linear chain, branched chain, or cyclic fatty acid.

### Composition for Iontophoresis

A composition useful in intradermally delivering a protein to an organism by iontophoresis comprises a negatively-charged protein-liposome complex, wherein the protein-liposome complex is formed of a negatively-charged protein and a cationic liposome. When the negatively-charged complex is administered to the skin of an organism by iontophoresis, although the complex has a larger molecular weight than that of the negative-charged protein, the complex efficiently permeates intradermally by crossing over a barrier of the skin. A composition useful in intradermally administering a protein to an organism by iontophoresis may comprise components in addition to the negatively-charged protein-liposome complex.

### Protein-liposome Complex

According to at least one embodiment, a protein-liposome complex is formed of a negatively-charged protein and a cationic liposome, and the net charge of the whole complex is negative. The protein-liposome complex may be formed by placing the protein and the liposome in a system capable of generating a charge interaction and aggregating the protein and the liposome to form the protein-liposome complex. In general, the protein-liposome complex is formed by binding of the protein and the liposome using an electrostatic interaction as a main driving force.

A negatively-charged protein-liposome complex has a zeta potential of preferably from −50 to −5 mV and more preferably from −40 to −10 mV.

A negative/positive (−/+) charge ratio of the negatively-charged protein to the positive charge of the cationic liposome may be appropriately determined in view of formation efficiency of the complex, and is preferably from 2:1 to 10:1, and more preferably from 3:1 to 8:1. The charge ratio is calculated based on the following principal: a half of the total charge of the liposome is used as the charge of the liposome because the liposome is formed of a lipid bilayer membrane and the positive charge inside the bilayer membrane is not involved in electrostatic interaction. For example, in the case where the liposome is formed of a monovalent cationic lipid having a positive charge, the −/+ ratio can be calculated by the following equation.

(−/+charge ratio)=[(protein amount (mol))×(total number of negative charges in protein)]:[cationic lipid amount (mol))/2] [Equation 1]

The protein amount and the cationic lipid amount can be easily determined in view of a loading amount and the like.

The average particle diameter of the protein-liposome complex is not particularly limited as long as a protein can be delivered intradermally by iontophoresis, and is preferably from 100 to 10,000 nm, and more preferably from 1,000 to 10,000 nm. As a method of determining the average particle diameter, there are given dynamic light scattering, static light scattering, observation with an electromicroscope, and observation with an atomic force microscope. Even if the protein-liposome complex has the particle size as described above, the complex may be administered to an organism intradermally by iontophoresis and has an advantage in administering a large protein, such as an antigen protein, intradermally to an organism.

### Negatively-charged Protein

A negatively-charged protein useful in a protein-liposome complex forms the complex in combination with the cationic liposome in a system capable of generating an interaction of charge.

The negatively-charged protein is not particularly limited as long as the protein is negatively charged and can form a complex with a liposome and is negatively charged, preferably, in a pH of from 3 to 10, more preferably in a pH of from 4 to 9.

A negatively-charged protein may be a protein formed of the same amino acids each having a negative charge and may be a protein formed of two or more different amino acids each having a negative charge. In addition, the protein may include both amino acids each having a positive charge and amino acids each having a negative charge as long as the protein has a net negative charge.

The total number of negative charges in the negatively-charged protein is preferably from 1 to 100 and more preferably from 5 to 100. The total number of the negative charges can be determined by one skilled in the art by the difference between the number of amino acids each having a negative charge and the number of amino acids each having a positive charge in the protein.

The molecular weight of the negatively-charged protein is not particularly limited as long as the negatively-charged protein can be delivered intradermally by the protein-liposome complex and is preferably from 3,000 kDa to 100,000 kDa and more preferably from 5,000 kDa to 50,000 kDa. Even a protein having the above molecular weight can be delivered. Accordingly, a protein-liposome complex can be advantageously used for delivering a protein having a relatively large molecular weight, such as an antigen protein.

A negatively-charged protein can preferably function as an antigen. Examples of a negatively-charged protein include, but are not particularly limited to, ovalbumin (hereinafter referred to as “OVA”), GAG protein of human immunodeficiency virus (HIV), hemagglutinin and neuraminidase of human influenza virus, and HBs antigen protein of hepatitis B virus.

### Cationic Liposome

A cationic liposome useful in a protein-liposome complex has a net positive charge and can form a negatively-charged protein-liposome complex in combination with a negatively-charged protein.

The average particle diameter of the cationic liposome is not particularly limited as long as the protein can be delivered intradermally, and is preferably from 50 to 1,000 nm and more preferably from 100 to 500 nm. A method of determining the average particle diameter is the same as the method of determining the particle diameter of the complex.

A liposome having a net positive charge comprises at least a cationic lipid as a constitutional component. The cationic lipid is preferably a C12-C20 lipid having a positive charge of from 1 to 10 valences, more preferably a C14-C20 lipid having a positive charge of from 1 to 3 valences, and more preferably a C14-C18 lipid having a positive charge of 1 valence. Specific examples of a cationic lipid include, but are not limited to, 1,2-dioleoyloxy-3-(trimethylammonium)propane (DOTAP), dioctadecyldimethyl ammonium chloride (DODAC), N-(2,3-dioleyloxy)propyl-N,N,N-trimethyl ammonium (DOTMA), didodecylammonium bromide (DDAB), 1,2-dimyristoyloxypropyl 1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Preferred are 1,2-dioleoyloxy-3-(trimethylammonium)propane (DOTAP), dioctadecyldimethyl ammonium chloride (DODAC), N-(2,3-dioleyloxy)propyl-N,N,N-trimethyl ammonium (DOTMA), and 2,3-dioleoyloxy-N-[2(spermine carboxamide)ethyl]-N,N-dimethyl-1-propanamium trifluoroacetate (DOSPA). More preferred is 1,2-dioleoyloxy-3-(trimethylammonium)propane (DOTAP).

In addition, the liposome preferably further comprises a sterol, a phospholipid, or a combination thereof.

A sterol may be appropriately selected in view of the stability of the cationic liposome, the delivery efficiency of the protein-liposome complex, and the like, and is preferably cholesterol (Chol), a cholesteryl fatty acid, a dihydrocholesteryl fatty acid, or a cholesteryl ether, and more preferably cholesterol (Chol), a C12-C31 cholesteryl fatty acid, a C12-C31 dihydrocholesteryl fatty acid, polyoxyethylene cholesteryl ether, and a polyoxyethylene dihydrocholesteryl ether, and still more preferably cholesterol (Chol).

A phospholipid may be appropriately selected in view of the delivery efficiency of the protein-liposome complex and is preferably a C12-C20 phospholipid and more preferably a C14-C18 phospholipid. Specific examples of a phospholipid include, but are not limited to, phosphatidyl choline, egg phosphatidylcholine (EPC), dipalmitoyl phosphatidyl choline (DPPC), and phosphatidyl ethanol amine. Preferred is distearoyl L-a-phosphatidylcholine (DSPC), egg phosphatidylcholine (EPC), dipalmitoyl phosphatidyl choline (DPPC), or combinations thereof. More preferred is distearoyl L-α-phosphatidylcholine (DSPC).

In addition, in the case where the liposome comprises a phospholipid, a sterol, or a combination thereof in addition to the cationic lipid as a constitutional component, the molar ratio of the cationic lipid to a sum of the phospholipid and the sterol may be appropriately determined in view of intradermal delivery efficiency or the like, and is preferably from 1:9 to 9:1 and more preferably from 2:8 to 8:2. Further, in the case where the liposome comprises both the phospholipid and the sterol, the molar ratio of the phospholipid to the sterol is preferably from 2:8 to 8:2 and more preferably from 3:7 to 7:3. In addition, according to one embodiment, the molar ratio among the cationic lipid, the phospholipid, and the sterol is about 2:5:3.

A protein-liposome complex can be used as a composition for iontophoresis in the methods disclosed herein. The composition may comprise one or more components in addition to the protein-liposome complex as long as the additional components do not prevent administration of the protein-liposome complex by iontophoresis.

The additional components are not particularly limited as long as the additional components do not prevent the administration of the protein-liposome complex by iontophoresis. Examples of additional components include, but are not limited to, water and pharmaceutically acceptable carriers such as buffering agents such as HEPES, preservatives, solubilizing agents, antiseptic agents, stabilizers, antioxidants, and colorants.

A composition comprising a protein-liposome complex may be formed into an appropriate formulation as desired as long as administration of the protein-liposome complex by iontophoresis is not prevented. For example, a composition comprising a protein-liposome complex can be formed into a dried form. However, in view of efficient administration of the complex by iontophoresis, the composition is preferably formed into a solution in combination with water or a HEPES buffer. In this case, the pH of the composition for iontophoresis is from 7 to 8, for example. In addition, the ion strength of the composition is from 5 to 20 mM, for example.

The content of the protein-liposome complex in the composition may be appropriately determined as required.

### Production Method

A protein-liposome complex can be easily formed by mixing a protein and a cationic liposome and aggregating the mixture.

For example, a liposome for use in forming a protein-liposome complex is prepared. The liposome may be prepared by methods known to one skilled in the relevant art or by the methods disclosed herein.

For example, a cationic lipid, a phospholipid, and a sterol are mixed at a desired ratio in a liquid medium such as water, whereby a mixed solution is obtained. Next, the medium is removed under reduced pressure, and thus a lipid membrane is obtained. Next, a buffer, for example HEPES buffer (10 to 50 mM), is added to the lipid membrane. The obtained mixed solution is left to stand at room temperature for about 10 minutes to hydrate the mixed solution, followed by sonication. Conditions for the sonication are, for example, but not limited to: 85 W, room temperature, and about 1 minute. Further, the mixed solution is treated with a membrane filter, an extruder, or the like to adjust the particle diameters as required, whereby the liposome is obtained.

Next, a first aqueous solution containing a negatively-charged protein and a second aqueous solution containing the liposome are prepared.

The concentration of the negatively-charged protein in the first aqueous solution and the concentration of the liposome in the second aqueous solution are appropriately determined in view of solubility of each protein and liposome to the solvent, formation efficiency of the negatively-charged protein-liposome complex, and the like.

The pH, ion strength, and temperature of the first and second aqueous solutions may be appropriately adjusted in view of the charging states of the protein and the liposome, and formation efficiency of the final complex.

Solvents used in the first and second aqueous solutions are preferably water and buffers, and more preferably water, HEPES buffer, and the like.

The first aqueous solution and the second aqueous solution are mixed together to obtain a mixed liquid. The mixing method is not particularly limited. The second aqueous solution may be added to the first aqueous solution, and alternatively, the first aqueous solution may be added to the second aqueous solution. In addition, the first aqueous solution and the second aqueous solution may be added into a container simultaneously to be mixed. The obtained mixed liquid of the first aqueous solution and the second aqueous solution may be stirred appropriately.

The mixing ratio of the first and the second aqueous solutions may be appropriately determined so that the protein-liposome complex having excess negative charge is formed in view of the −/+ charge ratio of the negatively-charged protein and the cationic liposome in the mixed liquid. For example, the mixing ratio may be set so that the total number of the negative charges in the first aqueous solution is in excess of the total number of positive charges in the second aqueous solution. In addition, the number of charges in both solutions can be easily set in view of the molar ratio of the protein or the cationic lipid, which is used in the preparation of both solutions, and the net number of charges.

In addition, the pH, ion strength, and temperature of the mixed liquid may be appropriately determined in view of the formation efficiency of the complex. The pH and ion strength of the mixed liquid can be adjusted by changing the compositions (e.g., concentration, amount, pH, and ion strength) and the mixing ratios of the first aqueous solution and the second aqueous solution beforehand. Specifically, the pH of the mixed liquid is from 3 to 10, for example. In addition, the ion strength of the mixed liquid is from 5 to 20 mM, for example.

The temperature of the mixed liquid is from 16° C. to 40° C., for example.

The mixed liquid may be left to stand as it is and the protein-liposome complex allowed to generate therein, wherein the mixed liquid can be used as a composition for iontophoresis. The mixed liquid is preferably incubated before the formation of the protein-liposome complex therein.

Conditions for incubating the mixed liquid may be for example, but not limited to, from 16° C. to 40° C.; and from 15 to 60 minutes.

The incubated mixed liquid may be centrifuged to obtain an aggregation. Conditions for the centrifugation may be, for example, but not limited to, 3,000 to 10,000×g; from 2° C. to 6° C.; and from 5 to 10 minutes.

An aqueous solvent, for example, but not limited to, water or a buffer, is added to the obtained aggregation, whereby a composition for iontophoresis containing a negatively-charged protein-liposome complex can be obtained. The composition for iontophoresis can be adjusted by one skilled in the relevant art to have a favorable pH and ion strength suitable for a negatively-charged complex with a buffer or the like. In this case, in view of the zeta potential of the negatively-charged complex or the like, the pH and the ion strength are preferably adjusted.

The temperature of the aqueous solvent may be appropriately determined by one skilled in the relevant art according to the formation efficiency of the protein-liposome complex, and is, for example, from 16° C. to 40° C.

### Application

The protein-liposome complex, and compositions thereof, is applied to an organism by iontophoresis and preferably used as a vaccine to induce an immune response in an organism. Further, a protein-liposome complex, and compositions thereof, can be used in iontophoresis to efficiently deliver intradermally a protein having antigenicity to an organism, wherein the immune response of the organism is effectively induced. Accordingly, a protein-liposome complex and compositions thereof, are preferably used as a intradermal vaccine preparation.

### Electrode Assembly and Iontophoresis Device

In addition, administration of the protein-liposome complex to an organism can be favorably performed by using an electrode assembly which holds the composition for iontophoresis and an iontophoresis device equipped with the electrode assembly.

According to one embodiment, provided is an electrode assembly including an electrode, a protein-liposome complex holding portion for holding the composition, the holding portion being placed in a skin side of the electrode, in which the protein-liposome complex can be released to an organism skin by iontophoresis. In addition, an electrolyte solution holding portion may be further provided between the electrode and the protein-liposome complex holding portion as long as administration of the protein-liposome complex by iontophoresis is not prevented.

In addition, the protein-liposome complex is charged negatively, so a negative current is preferably applied to a cathode side of an electric system. Therefore, in the electrode assembly of such an embodiment, the electrode refers to a cathode electrode. In addition, as the electrode, an electrode formed of a conductive material such as carbon or platinum is preferably used. Those materials can be also used in the counter electrode as described below.

The protein-liposome complex holding portion may be formed of a cell (electrode chamber) made of acryl or the like, which is impregnated with, holds, and is filled with the composition. Alternatively, the protein-liposome complex holding portion may be formed of a non-woven fabric, an absorbent cotton, or a thin membrane body, which is impregnated with and holds the composition. As a constitutional member of the thin membrane body, a material having both a favorable impregnation-holding characteristic and a favorable ion delivery property is preferred. Examples of the material include a hydrogel body of an acrylic resin (acrylic hydrogel membrane) and a segmented polyurethane-based gel membrane. The above cell and thin membrane body can be also used in the constitution of the electrolyte solution holding portion.

In addition, the constitution of the iontophoresis device can be appropriately changed as long as the iontophoresis device includes the electrode assembly and is capable of administering the protein-liposome complex. The iontophoresis device preferably includes at least an electric source unit, the electrode assembly connected to a cathode of the electric source unit, and an electrode assembly connected to an anode of the electric source unit.

The structure of the electrode assembly connected to the anode can be appropriately changed as long as the electrode assembly can function as a counter electrode of the electrode assembly which holds the protein-liposome complex. For example, the electrode assembly as a counter electrode may have the same constitution as that of the electrode assembly connected to an anode except that the protein-liposome complex holding portion is changed to the electrolyte solution holding portion. According to one embodiment, the electrode assembly as a counter electrode includes at least an anode electrode. In addition, according to a preferred embodiment, the electrode assembly as a counter electrode further includes the electrolyte solution holding portion which is provided to a skin side of the anode electrode and holds an electrolyte solution.

### Method of Intradermally Administering a Protein/Method of Inducing the Immune Response in an Organism

A composition comprising a protein-liposome complex can remarkably induce the immune response of an organism to the protein by intradermally administering the composition to the organism with the above iontophoresis device or the like. Therefore, according to another embodiment, provided is a method of inducing the immune response of an organism to a protein, the method comprising intradermally administering to an organism by iontophoresis an effective dose of a negatively-charged protein-liposome complex formed of a protein and a cationic liposome.

In the iontophoresis, energization conditions may be appropriately determined in view of administration efficiency of the protein-liposome complex. The current value is preferably from 0.1 to 0.45 mA/cm2 and more preferably from 0.1 to 0.2 mA/cm2.

In addition, in the method of inducing immune response in an organism, an effective dose of an adjuvant is preferably further administered to the organism in view of improving the immune response of the organism to a protein. The adjuvant may be administered sequentially to the organism before or after the iontophoresis, or may be administered with the protein-liposome complex simultaneously. The method of administering the adjuvant is not particularly limited as long as the method does not prevent the effect of the adjuvant. For example, there is a method of applying an adjuvant to the portion of skin of the organism to which the iontophoresis is applied.

Adjuvants are known to one skilled in the relevant art. Preferred adjuvants are intradermal adjuvants. More preferred adjuvants are monophospholipid A, oligo nucleic acid sequence (so-called CpG), lipopolysaccharide (so-called LPS), muramyl dipeptide (MDP), Mycobacterium bovis cell wall (BCG-CWS), and the like. More preferred is monophospholipid A.

The dose of the protein-liposome complex or the adjuvant effective to induce an immune response in an organism is appropriately determined by a person skilled in the art in view of the kind of disease, species, gender, age, body weight, and state of the organism, administration plan, and the like.

An “organism” is preferably a mammalian animal, more preferably a human, cow, pig, horse, sheep, dog, or cat, and still more preferably a human.

Hereinafter, embodiments of protein-liposome complexes, compositions comprising protein-liposome complexes and methods of intradermal administration thereof by iontophoresis are described in detail by way of examples. It is understood that the invention is not limited by these examples.

### Example 1

### 1. Fluorescent Labeling of OVA

A solution containing 10 mg of OVA (SIGMA Aldrich) in a borate buffer solution and 2.81 mg of NHS-Rhodamine dissolved in 100 μl of DMF were mixed, followed by reaction at room temperature for 1 hour. The obtained mixed liquid was subjected to a gel filtration using Sephadex-G100 to separate free NHS-Rhodamine, whereby a Rhodamine-labeled OVA was obtained.

### 2. Preparation of OVA-liposome Complex

DOTAP, DSPC, and Chol were mixed into an organic solvent such as CHCl₃ at a ratio of 2/5/3 (DOTAP/DSPC/Chol), whereby a solution (total lipid weight of 1.6 mg) was obtained. The organic solvent was removed under reduced pressure, and the addition of the organic solvent and the removal of the organic solvent under reduced pressure were repeated to yield a lipid thin membrane. Next, 0.5 ml of 10 mM HEPES buffer was added to the lipid thin membrane so that the total concentration of the lipid was 5 mM, followed by hydration at room temperature for 10 minutes. Next, the resulting mixture was sonicated in a bath-type sonicator, and a liposome (hereinafter referred to as “DSPC liposome”) solution was obtained.

150 μl of a 100 mg/ml OVA aqueous solution prepared by using the Rhodamine-labeled OVA was added to 500 μl of the DSPC liposome solution. The obtained mixed liquid was incubated at room temperature for 30 minutes and centrifuged at 5,000×g and 4° C. for 5 minutes. The obtained pellet was suspended in 150 μl of a 10 mM HEPES buffer, whereby an OVA-liposome complex solution was obtained.

### Reference Example 1

### Preparation of OVA-encapsulated Liposome

DOTAP, egg phosphatidylcholine (hereinafter referred to as “EPC”), and Chol were mixed in an organic solvent such as CHCl₃ at a ratio of 4/4/2 (DOTAP/EPC/Chol), whereby a solution (total lipid weight of 8.3 mg) was obtained. The organic solvent was distilled off under reduced pressure, and the addition of the organic solvent and the removal of the organic solvent under reduced pressure were then repeated to yield a lipid thin membrane. Next, 1 ml of acetate buffer (pH 4.5) containing 5 mg/ml Alexa 448-labeled OVA (Invitrogen) was added to the lipid thin membrane so that the total concentration of the lipid was 12.5 mM, followed by hydration at room temperature for 10 minutes. Next, after the resulting mixture was sonicated in a bath-type sonicator, the resulting mixture was frozen and thawed (6×) to yield a solution. After the solution was extruded using a 1,000-nm film, the resultant was then centrifuged at 80,000 g and 4° C. for 30 minutes to remove free OVA. The obtained pellet was suspended with 300 μl acetate buffer, whereby an OVA-encapsulated liposome solution was obtained.

### Test Example 1

The solution of the OVA-liposome complex or the OVA-encapsulated liposome were filled in a dedicated cuvet, whereby a particle diameter and a zeta potential were measured with a Zetasizer®.

The results are as shown in Table 1. The OVA-liposome complex was charged negatively, while the OVA-encapsulated liposome was charged positively. In this case, the −/+ charge ratio of the negative charge of OVA to the positive charge of liposome in the OVA-liposome complex was calculated to be 7:1 [(the number of moles of OVA×the number of negative charges):(the number moles of DOTAP×1/2)].

TABLE 1
Measurement results of particle diameter and zeta potential

| | Average particle | |
|---|---|---|
| | diameter (nm) | Zeta potential (mV) |
| OVA-liposome complex | 5,285 ± 4,240 | −20.06 ± 5.81 |
| OVA-encapsulated liposome | 545 ± 351 | 53.75 ± 6.01 |

*The numeric values in Table 1 represent average value ± standard deviation.

### Test Example 2

### Iontophoresis

The OVA-liposome complex or the OVA-encapsulated liposome were administered to an SD rat (10-week-old, male) by iontophoresis according to the following procedure.

### Iontophoresis Device

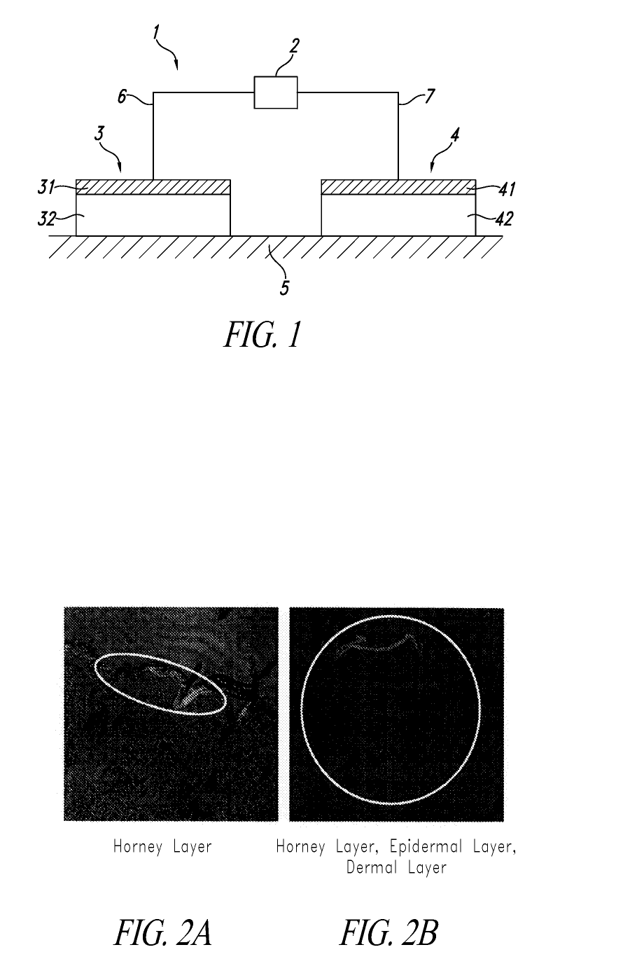

The OVA-liposome complex was charged negatively, thereby being administered from a cathode side. The iontophoresis device used in the administration of the OVA-liposome complex is as shown in FIG. 1.

In FIG. 1, an iontophoresis device 1 is placed on a skin 5 and is formed of a electric source unit 2, an electrode assembly 3 for holding the complex, and an electrode assembly 4 as a counter electrode thereof. Those electrode assemblies are connected with electrically conductive paths 6 and 7. The electrode assembly 3 was formed of a cathode electrode 31, a protein-liposome complex holding portion 32 provided at a skin side of the cathode electrode 31. On the other hand, the electrode assembly 4 was formed of an anode electrode 41, an electrolyte holding portion 42 which was provided at a skin side of the anode electrode 41 and held 200 μl of an electrolyte solution. In addition, the protein-liposome complex holding portion 32 and the electrolyte solution holding portion 42 each were formed of a non-woven fabric or absorbent cotton impregnated with an siRNA-polycation complex solution or an electrolyte solution.

In addition, in the case where the OVA-encapsulated liposome was administered, the OVA-encapsulated liposome was charged positively, thereby being administered from the anode side.

### Administration of OVA-liposome Complex

An antigen holding portion of the iontophoresis device was filled with 200 μl of the OVA-liposome complex solution of Example 1. Next, an iontophoresis device was mounted on a back skin of an SD rat (10-week-old, male) which had been shaved with clippers under Nembutal® anesthesia. Then, iontophoresis was performed at a constant current of 0.45 mA (0.15 mA/cm²) for 60 minutes.

### Administration of OVA-encapsulated Liposome

An antigen holding portion (at the anode side) of the iontophoresis device was filled with 200 μl of the OVA-encapsulated liposome solution of Reference Example 1. Next, an iontophoresis device was mounted on a back skin of an SD rat (10-week-old, male) which had been shaved with clippers under Nembutal® anesthesia. Then, iontophoresis was performed at a constant current of 0.45 mA (0.15 mA/cm²) for 60 minutes.

### Preparation of Skin Slice and CLSM Observation

The skin was cut out 3 hours after the energization, and the resultant was frozen and embedded in an Optimal Cutting Temperature (OCT) compound. A slice measuring 20 μm was prepared from the frozen skin sample with a cryostat and observed with an inverted confocal laser scanning microscope (LSM510 Carl Zeiss).

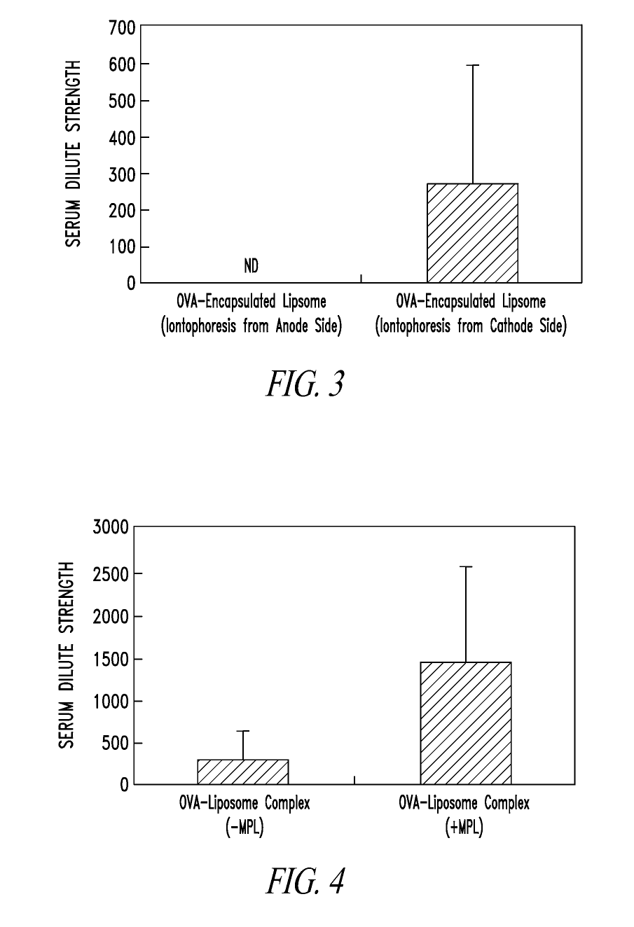

As a result, as shown in FIG. 2A, in the case where the OVA-encapsulated liposome was administered from the anode side, little fluorescence was observed in the epidermal layer or the dermal layer except in a horny layer. On the other hand, in the case where the OVA-liposome complex was administered from the cathode side, as shown in FIG. 2B, fluorescence was observed in the epidermal layer and the dermal layer.

### Test Example 3

### Preparation of OVA-encapsulated Liposome and OVA-liposome Complex

An OVA free from fluorescent labeling was used and an OVA-encapsulated liposome and an OVA-liposome complex were prepared by the same procedure as in Example 1 and Reference Example 1.

### Transdermal Immunity by Iontophoresis

The prepared OVA-encapsulated liposome or the OVA-liposome complex was administered to a back skin of an SD rat by iontophoresis in the same procedure as in Test Example 2. The OVA-encapsulated liposome or the OVA-liposome complex was administered by iontophoresis again in the same procedure 1 week after the first administration. Blood was sampled 1 week after the second administration and serum was collected by centrifugation.

### Evaluation for Anti-OVA Antibody Amount by ELISA Method

The production amount of the anti-OVA antibody in the obtained serum was evaluated by an ELISA method according to the following procedure.

0.01 mg/ml OVA dissolved in a carbonate buffer (pH 9.5) was added to a 96-well microtiter plate (nunc immunoplate maxisorp corting) at 50 μl/well, followed by incubation at 4° C. overnight. Next, by using Immunowash™ (BIO-RAD™), the plate was washed with a wash buffer (−) (1 L PBS+2.1 g NaCl) three times and a blocking agent (1% casein+1% gelatin+0.5% BSA carbonate buffer solution) was added at 150 μl/well, followed by incubation at 37° C. for 1 hour. After that, the plate was washed with wash buffer (+) (wash buffer (−)+0.05% triton-X) in the same manner as described above three times. Then, serum samples each diluted step by step 10-fold to 20.000-fold were added to the wells. After incubation at 37° C. for 1 hour, the plate was washed with wash buffer (+) three times in the same manner as described above. 50 μl of an HRP-labeled secondary antibody was added to each well. After incubation at 37° C. for 1 hour, the plate was washed with wash buffer (+) three times in the same manner as described above, and 50 μl of TMB was added to each well to cause color developing. After incubation at room temperature for 10 minutes, 50 μl of 1 N sulfuric acid were added to each well to stop color developing. Absorbance was measured at 450 nm with a microplate reader (BIO-RAD™).

The production amount of the anti-OVA antibody was evaluated by subtracting the absorbance value in the serum of an untreated rat from the measured absorbance value, and by using as an index a dilute strength of the serum sample which is equal to or more than a detection limit (absorbance of 0.08).

The results are as shown in FIG. 3. In the case where the OVA-encapsulated liposome was administered from the anode side, the anti-OVA antibody in the serum was not detected at all dilute strength at all (ND:not detect). On the other hand, in the case where the OVA-liposome complex was administered from the cathode side, the anti-OVA antibody was detected even at about 300-fold dilute strength (average value±standard deviation), whereby it was confirmed that the antibody production was induced.

### Test Example 4

### 1. Preparation of OVA-liposome Complex

An OVA free from fluorescent labeling was used and an OVA-liposome complex was prepared by the same procedure as in Example 1.

### 2. Transdermal Immunity by Iontophoresis in Combination with Monophospholipid A (MPL)

After 100 μl of a commercially available MPL adjuvant was coated on a back skin of a rat which had been shaved with clippers, the OVA-liposome complex was administered by iontophoresis in the same procedure as in Test Example 2. The same test was performed without coating MPL as a control. Blood was sampled one week after the second iontophoresis and serum was collected by centrifugation.

Evaluation for Production Amount of anti-OVA Antibody by ELISA Method

The production amount of the antibody was measured and evaluated by an ELISA method according to the same procedure in Test Example 3.

The results are as shown in FIG. 4. The production amount of the antibody (average value) in the case where MPL was coated to an antigen-administered portion immediately before iontophoresis was about 4 times that in the case where MPL was not used. In addition, in the case of coating MPL, even if the serum was diluted 1.000-fold or more, the anti-OVA antibody was significantly detected.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without departing from the spirit and scope of the disclosure, as will be recognized by those skilled in the relevant art. The teachings provided herein of the various embodiments can be applied to other delivery methods, not necessarily the exemplary iontophoretic intradermal delivery method generally described above.

The various embodiments described above can be combined to provide further embodiments. To the extent that they are not inconsistent with the specific teachings and definitions herein, all of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ systems, circuits and concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

I/we claim:

1. A composition for iontophoresis, comprising:
a protein-liposome complex which is charged negatively, wherein the protein-liposome complex is formed of a negatively-charged protein and a cationic liposome.

2. The composition according to claim 1 wherein the negatively-charged protein and the cationic liposome are bound to each other by an electrostatic interaction.

3. The composition according to claim 1 wherein the protein-liposome complex has a zeta potential of −50 to −5 mV.

4. The composition according to claim 1 wherein a negative to positive (−/+) charge ratio of the negatively-charged protein to the cationic liposome is from 2:1 to 10:1.

5. The composition according to claim 1 wherein the protein-liposome complex has an average particle diameter of from 100 nm. to 10,000 nm.

6. The composition according to claim 1 wherein the negatively-charged protein has a molecular weight of from 3,000 kDa to 100,000 kDa.

7. The composition according to claim 1 wherein the negatively-charged protein has a total number of negative charges of from 1 to 100.

8. The composition according to claim 7 wherein the cationic lipid is a C12-C20 lipid having a positive charge of from 1 valences to 10 valences.

9. The composition according to claim 1 wherein the protein has a negative charge at a pH of from 3 to 10.

10. The composition according to claim 1 wherein the negatively-charged protein has antigenicity.

11. The composition according to claim 1 wherein the cationic liposome has an average particle diameter of from 50 nm. to 1,000 nm.

12. The composition according to claim 1 wherein the cationic liposome comprises a cationic lipid.

13. The composition according to claim 12 wherein the cationic lipid is 1,2-dioleoyloxy-3-(trimethylammonium)propane, dioctadecyl dimethyl ammonium chloride, N-(2,3-dioleoyloxy)propyl-N,N,N-trimethyl ammonium, didodecylammonium bromide, 1,2-dimyristoyloxypropyl 1,3-dimethylhydroxyethyl ammonium, or 2,3-dioleoyloxy-N-[2(spermine carboxyamide)ethyl]-N,N-dimethyl-1-propanamium trifluoroacetate.

14. The composition according to claim 12 wherein the cationic liposome further comprises a sterol, a phospholipid, or a combination thereof.

15. The composition according to claim 12 wherein the sterol is cholesterol, a cholesteryl fatty acid, a dihydrocholesteryl fatty acid, or a cholesteryl ether.

16. The composition according to claim 15 wherein the sterol is cholesterol.

17. The composition according to claim 14 wherein the phospholipid is a C12-20 phospholipid.

18. The composition according to claim 14 wherein the phospholipid is selected from the group consisting of distearoyl L-a-phosphatidylcholine, egg phosphatidylcholine, and dipalmitoyl phosphatidylcholine.

19. The composition according to claim 1 which is in a dry form.

20. The composition according to claim 1 which is used as a drug.

21. A vaccine preparation, comprising:
a composition comprising a protein-liposome complex which is negatively charged and which is formed of a negatively-charged protein and a cationic liposome, the negatively-charged protein having an antigenicity.

22. The vaccine preparation according to claim 21 which is used with an adjuvant.

23. The vaccine preparation according to claim 22 wherein the adjuvant is a transdermal adjuvant.

24. The vaccine preparation according to claim 23 wherein the transdermal adjuvant is selected from the group consisting of monophospholipid A, oligo nucleic acid sequence, lipopolysaccharide, muramyl dipeptide, and Mycobacterium bovis cell wall.

25. An electrode assembly for iontophoresis, comprising:
an electrode; and
a protein-liposome complex holding portion for holding the composition according to claim 1, the holding portion being provided in a skin side of the electrode, wherein
the electrode assembly is capable of releasing a protein-liposome complex to an organism skin by iontophoresis.

26. The assembly according to claim 25, further comprising:
a counter electrode; and
an electric power unit, wherein the electrode is electrically coupled to a cathode of the electric power unit and the counter electrode is electrically coupled to an anode of the electric power unit.
